(19) United States
(12) Patent Application Publication
(10) Pub. No.: US 2009/0177228 A1
(43) Pub. Date: Jul. 9, 2009

(54) Coated suture thread and production thereof

(75) Inventors: Per Vilhelm Aspenberg, Linköping (SE); Pentti Tengvall, Linköping (SE); Björn Pasternak, Linköping (SE)

Correspondence Address: BACON & THOMAS, PLLC, 625 SLATERS LANE, FOURTH FLOOR, ALEXANDRIA, VA, 22314-1176, US

(73) Assignee: Optovent AB, Linkoping (SE)

(21) Appl. No.: 11/921,025

(22) PCT Filed: Feb. 21, 2006
(86) PCT No.: PCT/SE2006/000228

§ 371 Date: Feb. 13, 2009

Related U.S. Application Data
(63) Continuation of application No. 11/138,719, filed on May 26, 2005, now abandoned.
Application No. 11/138,719 is a continuation-in-part of application No. 10/534,666, filed on May 11, 2005, now Pat. No. 7,163,690.

(51) Int. Cl. A61B 17/04; B05D 3/00

(52) U.S. Cl. 606/228; 427/2.31

(57) ABSTRACT

Disclosed is a coated suture thread comprising a matrix formed by an immobilized and crosslinked plurality of fibrinogen layers into and/or onto which one or several pharmacological substances that inhibit tissue break-down, such as MMP inhibitors and/or corticosteroids and/or COX inhibitors, are attached and/or associated. Further, a method of producing such a coated suture thread as well as the use thereof for suturing damaged tissue, such as damaged tendon, ligament, intestine and/or skin, are described.

The present invention relates to coated suture threads intended for use in human as well as animal subjects to inhibit tissue breakdown around the suture threads. The invention relates also to a method of producing suture threads coated with crosslinked fibrinogen and pharmacological substances that inhibit tissue break-down, such as a matrix metallo-proteinase inhibitor (MMP-inhibitor) and/or a corticosteroid and/or a cyclooxygenase inhibitor (COX-inhibitor).

### BACKGROUND OF THE INVENTION

Collagen is the fundamental functional molecule in tissues like tendon and ligaments, and is largely responsible for the mechanical integrity of most tissues in the body, ranging from intestines to tendons. When a ligament or tendon has ruptured, or when other organs are operated on, the repair is often done with sutures. The suture threads have a grip in the collagenous substance of the tissue. The cells in the vicinity of the suture become activated by the trauma, either by the injury or by the presence of the suture itself. This activation leads to break-down of the tissue. It is well known that, for example, the repair of a ruptured tendon or an opened intestine with a suture will have a decreasing mechanical strength during the first days or weeks. Even though the suture fixation appears strong at the time of the operation, there is a high risk that the suture thread will cut through the tendon material when this has become softened in the days following the operation. This softening of the tissue around the sutures is a real problem that has troubled medical practitioners for a long time.

It is well known that tendon suture fixation is imperfect. Although elaborate suture techniques improve tensile strength of tendon repair, all appear to result in decreasing suture fixation some days after the operation. Studies concerning flexor or Achilles tendon repair report a (re)rupture rate of 3 to 6%. Repair-site elongation (gap formation) is much more common, resulting in slower healing and with that, poorer clinical outcome.

Matrix metalloproteinases (MMPs) are essential in tissue remodelling and act as part of the inflammatory response to clear up debris. Implantation of a foreign material into the tendon invariably evokes a tissue reaction and there is evidence of elevated MMP activity in the direct vicinity of sutures inserted into tendons.¹ This probably weakens the tissue and allows the suture to cut through the tendon when exposed to tensile stress, i.e. in early mobilisation.

Matrix Metallo-Proteinases (MMPs) are a group of zinc-dependent enzymes responsible for the breakdown of collagen and other matrix molecules. MMPs are crucial in the turn-over of tissue matrix. A dramatic increase in the production and activation of MMPs is caused by injury or surgery on collagenous tissues. This leads to break-down of tissue and reduced strength following the suture procedure. This over-production of MMPs leads to break-down of tissue and reduced strength of the substances disposed around the sutures.

The suture threads themselves may also cause increased production of MMP. There is evidence that cells produce large amounts of MMPs specifically around sutures inserted into tendons. Additionally, unloading the tendon, such as in a cast after injury or surgery, can lead to dramatic deterioration of its mechanical properties within a short period of time. Immobilization, in both tendons and ligaments, leads to an increase in local MMP production. Thus, the weakening of tendons after injury, suture and unloading should principally be the result of increased MMP activity. The clinical problems corresponding to these phenomena are repair-site elongation, implying poorer healing, and, at worst, re-rupture.

Rates of anastomotic leakage are significant after suturing intestines (intestinal anastomoses). Studies show that MMP production (expression) is high, especially at the suture line. It has recently been demonstrated that suturing of the bowel (colon) in rats is followed by a fast decrease in suture strength by about 50%. Systemic treatment of the rat with a metallo-proteinase inhibitor totally abolished this decrease in strength.

However, potent MMP-inhibitors administered systemically can cause joint stiffness and swelling and possibly other toxic reactions². Additionally, there are concerns about detrimental effects of MMP-inhibitors on cutaneous wound healing³. Hence, local delivery of an MMP-inhibitor in humans would be advantageous over systemic administration.

Anastomotic leakage is a major and unresolved problem in patients undergoing colonic or rectal resection⁴. Under experimental conditions, the strength of intestinal anastomoses diminishes postoperatively reaching minimum on the third postoperative day⁵. Increased matrix metalloproteinase (MMP) activity is thought to mediate the loss of anastomotic strength by causing local matrix degradation in the tissue surrounding the sutures. Several MMPs, e.g. collagenase 2 (MMP-8), gelatinase B (MMP-9) and stromelysin 1 (MMP-3), are upregulated in the direct vicinity of the anastomotic suture line^{6,7}

Thus, MMP inhibitors may be used to inhibit the breaking down of tissue caused by the MMPs. Skin wound healing can be accelerated by using MMP inhibitors. Animal models show that MMP-inhibitors enhance mechanical properties of (non-sutured) healing skin wounds. In some surgical situations, such as hernial surgery, a mesh of suture-like material is implanted to augment soft tissues and serve as a scaffold for scar formation. It is conceivable that there is a problem with local MMP production and activation adjacent to this net in the same way as has been shown for ordinary suture threads.

Not only MMPs are activated in these situations. Cyclooxygenases are also activated which are enzymes responsible for the production of prostaglandins which are important for the development of inflammation. Inhibition of cyclooxygenases is done with common anti-inflammatory drugs. Such inhibition with systemic treatment improves the later phases of tendon repair.

Systemic medication, such as by administering substance in tablet or liquid form, implies drug interference with organs additional to those intended. Side-effects from systemic use of MMP-inhibitors for cancer therapy have for example been reported as well as from the antibiotic use of tetracyclines.

There is a need to inhibit the gradual weakening or softening of the tissue surrounding sutures that occurs after surgery. There is also a need to prevent the side effects of systemic distribution of the relevant drug to inhibit tissue weakening around the sutures.

### DESCRIPTION OF THE INVENTION

The present invention provides coated suture threads as well as a method of producing suture threads coated with crosslinked fibrinogen and pharmacological substances that inhibit tissue break-down. Fibrinogen is a flexible protein having 3 structurally bound calcium ions, and it can easily be used for building a matrix by crosslinking layers of fibrinogen. Further, it is suitable for the purpose of the invention since it has a low immunoactivation. Implementation of the present invention diminishes the decrease in tissue mechanical strength that is the result of injury, surgery and suturing. The method of the present invention is for coating pharmacological substances that inhibit tissue break-down onto a suture thread and the thus produced suture threads for use in surgery and suturing.

Thus, one aspect of the invention is directed to a coated suture thread comprising an immobilized and crosslinked fibrinogen matrix into and/or onto which one or several pharmacological substance(s) that inhibit tissue break-down is (are) attached and/or associated.

The immobilization of the matrix onto the suture thread surface may be by any suitable means such as covalent coupling, adsorption, van der Waals, hydrophobic, coulombic and/or other interactions.

Commercially available suture materials are suitable for use in the present invention e.g. Natural sutures, such as catgut, silk, collagen, cotton and linen; Synthetic non-adsorbable sutures, such as polyesters, polyamides, polypropylens, fluoropolymers and stainless steel; and Absorbable synthetic sutures such as poly(glycolic acid), poly(L-lactic acid), polydioxanone, poly(trimethylene carbonate), poly(caprolactone) and combinations thereof.

Examples of pharmacological substances that inhibit processes leading to tissue break-down and that can be included in the coatings of the suture threads of the invention are, but are not limited to:

a. Tetracyclines; e.g. doxycycline, oxytetracycline, tetracycline, minocycline, lymecycline.
b. Chemically modified tetracyclines (CMTs); e.g. CMT 3 (COL 3), CMT 8.
c. Synthetic matrix metalloproteinase inhibitors, including those of the hydroxamate subgroup; e.g. GM 6001 (ilomastat; N-[(2R)-2-(Hydroxamidocarbonylmethyl)-4-methylpentanoyl]-L-tryptophan methylamide), FN-439, GM 1498 (N-[(2R)-2-(Carboxymethyl)-4-methylpentanoyl]-L-tryptophan-(S)-methyl-benzylamide), CL-82198, AG3340 (prinomastat), BB-251 (marimastat); sulfonimidamides, such as sulfonimidamide hydroxamates.
d. Cyclooxygenase (COX) inhibitors, including cyclooxygenase 2 specific inhibitors; e.g. ibuprofen, diclofenac, naproxen, celecoxib, parecoxib, etoricoxib.
e. Nuclear factor kappa B inhibitors; e.g. 6-Amino-4-(4-phenoxyphenylethylamino) quinazoline, 4-Methyl-N¹-(3-phenylpropyl)benzene-1,2-diamine.
f. Lipooxygenase inhibitors; e.g. zileuton.
g. Corticosteroids including glucocorticoids; e.g. cortisone, dexamethasone, prednisolone, methylprednisolone, hydrocortisone.
h. Macrolide antibiotics; e.g. erythromycin, clarithromycin, azithromycin.
i. Hydroxymethylglutaryl coenzyme A reductase inhibitors (statins); e.g. simvastatin, atorvastatin, pravastatin, rosuvastatin.
j. Angiotensin converting enzyme (ACE) inhibitors; e.g. captopril, enalapril, ramipril.
k. Angiotensin II receptor blockers (ARBs); e.g. losartan, valsartan, kandesartan.
l. Bisphosphonates; e.g. pamidronate, alendronate, ibandronate, zolendronate, etidronate, risedronate.
m. Aprotinin
n. Gabexate mesilate
o. Sulfasalazine
p. Inhibitors of tumour necrosis factor alpha; e.g. infliximab, etanercept. These antibodies may be attached to the surface layer of fibrinogen since they will not penetrate into the fibrinogen matrix due to their size.
q. Transforming growth factor beta inhibitors; e.g. Transforming Growth Factor-β Type I Receptor Kinase Inhibitor ([3-(Pyridin-2-yl)-4-(4-quinonyl)]-1H-pyrazole), SB-431542.

It should be noted that the antibiotics on the list are not included because of their effect against bacteria, but because they are inhibitors of MMPs or other degradatory enzymes as well.

In a preferred embodiment of the suture thread of the invention the matrix is formed on a rough surface of the thread. The rough surface of the suture thread may have been obtained by mechanical, chemical or physical treatment.

In another preferred embodiment of the coated suture thread of the invention the fibrinogen matrix is covalently coupled to the suture thread surface, optionally via a linker molecule such as glutaraldehyde.

In an embodiment of the coated suture thread of the invention the fibrinogen matrix is built up of several layers selected from 2 to 20 layers, i.e. the total number of fibrinogen layers is 2, 3, 4, 5, 6, 7, 8, 9, 10, 11, 12, 13, 14, 15, 16, 17, 18, 19, or 20, but even more layers are possible to use even though it is not evident at present why more layers would be desired.

In another embodiment of the coated suture thread of the invention the one or several pharmacological substance(s) is (are) selected from the group consisting of tetracyclines, chemically modified tetracyclines, synthetic matrix metalloproteinase inhibitors, including those of the hydroxamate subgroup; cyclooxygenase inhibitors, including, cyclooxygenase 2 specific inhibitors; nuclear factor kappa B inhibitors; lipooxygenase inhibitors; corticosteroids, including glucocorticoids; macrolide antibiotics; hydroxymethylglutaryl coenzyme A reductase inhibitors (statins); angiotensin converting enzyme (ACE) inhibitors; angiotensin II receptor blockers (ARBs); bisphosphonates; aprotinin; gabexate mesilate; sulfasalazine; inhibitors of tumour necrosis factor alpha; and transforming growth factor beta inhibitors.

In a further embodiment of the coated suture thread of the invention the one pharmacological substance is a matrix metallo-proteinase inhibitor (MMP-inhibitor) and another one pharmacological substance is a corticosteroid and yet another one pharmacological substance is a cyclooxygenase inhibitor (COX-inhibitor).

It should be noted that the fibrinogen matrix on the suture thread may comprise several different pharmacological substances at the same time, such as different matrix metallo-proteinase inhibitors and/or different corticosteroids and/or different cyclooxygenase inhibitors. In this way it is possible to locally administer different drugs that can cooperate, even synergistically, in the inhibition of tissue break-down.

Another aspect of the invention is directed to a method of producing a coated suture thread comprising the steps of immobilizing a first layer of fibrinogen onto a suture thread surface to be coated,

crosslinking a second layer of fibrinogen to the first layer of fibrinogen to form a fibrinogen matrix,
optionally increasing the number of layers of the fibrinogen matrix by crosslinking one or several layers of fibrinogen on top of the second layer of fibrinogen, and
attaching and/or associating one or several pharmacological substance(s) that inhibit tissue break-down into and/or onto the matrix of immobilized and crosslinked plurality of fibrinogen layers.

The method of the invention can be practiced on any suture materials, such as the commercial materials mentioned above. Further, any pharmacological substances that inhibit processes leading to tissue break-down can be included in the coatings of the suture threads by the method of the invention, e.g. those listed above. In a preferred embodiment of this aspect of the invention the suture thread surface to be coated is roughened by mechanical, chemical or physical means to increase the surface area thereof. Braided threads are also possible to use. The increased surface area allows attachment of more fibrinogen to the surface and thus the possibility of carrying more drugs to the site of suture thread in the body. Further, a rough surface onto which the fibrinogen layer is attached, preferably conjugated, aids in resisting mechanical wear and/or abrasion of the coating from the thread when pulled through the tissue.

In another embodiment of this method aspect of the invention the surface of the suture thread to be coated is treated to generate chemically reactive groups thereon. In case radio frequency plasma treatment is used, both the reactive groups and the surface roughening are obtained.

In yet another embodiment of the method of the invention, the first fibrinogen layer is covalently coupled to the suture thread surface, e.g. by covalently coupling one end of a linker molecule, such as glutaraldehyde, to the suture thread surface and the other to the first layer of fibrinogen.

The number of crosslinked fibrinogen layers that can be immobilized onto the suture thread is not limited, but the total number of fibrinogen layers of the fibrinogen matrix produced is preferably selected from 2 to 20 layers as mentioned above.

In another embodiment of the method of producing a coated suture thread of the invention, the one or several pharmacological substance(s) that are attached and/or associated to the fibrinogen matrix is (are) selected from the group consisting of tetracyclines, chemically modified tetracyclines, synthetic matrix metalloproteinase inhibitors, including those of the hydroxamate subgroup; cyclooxygenase inhibitors, including cyclooxygenase 2 specific inhibitors; nuclear factor kappa B inhibitors; lipooxygenase inhibitors; corticosteroids, including glucocorticoids; macrolide antibiotics; hydroxymethylglutaryl coenzyme A reductase inhibitors (statins); angiotensin converting enzyme (ACE) inhibitors; angiotensin II receptor blockers (ARBs); bisphosphonates; aprotinin; gabexate mesilate; sulfasalazine; inhibitors of tumour necrosis factor alpha; and transforming growth factor beta inhibitors.

Presently preferred drugs to be included in the coating are a matrix metallo-proteinase inhibitor (MMP-inhibitor) a corticosteroid and a cyclooxygenase inhibitor (COX-inhibitor), each alone or in some combination.

The invention is also directed to a method of treating a subject in need of suturing damaged tissue, such as tendon, ligament, intestine and/or skin, comprising suturing the damaged tissue with a suture thread according to the invention

Thus, the present invention provides means for inhibiting tissue weakening. At the same time, the adverse effects of systemic distribution of the relevant medical substances are avoided. One important feature of the present invention is that a local drug delivery is achieved through the attachment of the relevant medical drug to the suture thread left in the body after a surgery or other intervention. Another important feature is to provide sustained medical treatment rather than merely a single dosage. Preferably, there is a gradual release of the locally delivered drug at the site of the surgery.

The present invention includes the coating of suture threads with enzyme inhibitors such as MMP-inhibitors or other substances that interfere with collagen and tendon tissue to contribute to the maintained integrity of collagen and tendon tissue. Preferably, the MMP inhibitors should be gradually released to reduce the effectiveness of MMP.

In general, MMP is over-produced in areas of injury or surgery so that the amount of tissue that is broken down exceeds the reproduction of tissue. By gradually releasing MMP inhibitors there is a better balance created in the area of injury between the breaking down of tissue by MMP and new reproduction of tissue. In this way, suture threads may be more effectively used in areas of injury or surgery since the tissue surrounding the suture threads is not broken down as much or not at all. More particularly, the MMP inhibitor should be gradually released as long as there is an injury so as to inhibit the effectiveness of the overproduced MMP during this time. When the injury has healed there is no need to inhibit MMP and the natural balance between the breaking down of tissue and regeneration of tissue is reestablished.

Adherence of the drug substance, containing MMP-inhibitors, to the suture or other device is achieved by adsorption, covalent binding, electrostatic interactions or any other suitable mechanism. Preferably, the adherence is achieved through the formation of a fibrinogen matrix, and the binding of MMP-inhibitor (or other substance) to the matrix to reduce the effect of MMP around the suture.

In this way, the MMP inhibitor coated suture has a fibrinogen matrix attached to it in which the MMP-inhibitor is associated. The matrix may be composed of several layers of fibrinogen covalently bound to each other, and the bottom layer may be attached via adsorption, covalent binding, van der Waals, hydrophobic, coulombic and/or other interactions, to the suture material. In case a weak binding is selected for the attachment, the fibrinogen matrix may be detached from the suture surface and form a sleeve or tube around the thread. Preferably, a first amount of the MMP inhibitor is covalently bound to the matrix. However, other ways of binding the inhibitor may be used such as electro-static binding or hydrophobic attachment of the inhibitor. A combination of covalent and electrostatic loading may be preferable to obtain a gradual release of the MMP inhibitor. Preferably, a second amount of the MMP inhibitor is adhered to the matrix for easy release. The gradual release of the MMP inhibitor, or other drugs, from the coated suture thread decreases the local MMP activity and thus preserves the mechanical strength of the collagenous material surrounding the suture thread and decreases the risk of failure of the sutured tissue. A COX-inhibitor could serve the same purpose, but acting in an earlier step, i.e. reducing the induction of MMP-producing activity of inflammatory and tissue cells and is therefore one of the possible drugs in the coating on the suture thread of the invention.

As the drugs, e.g. MMP inhibitors, are incorporated in a matrix adhered to the suture, the release velocity and also pharmacological doses can be controlled. Additionally, more substance per area suture can be delivered by using the matrix. This should be done in a way that permits a controlled release of the MMP inhibitor that last several days or even a few weeks. The incorporation of the drug within the substance of a resorbable thread, such as a PLGA suture, releases most of the drug too late, when the suture is losing strength in itself and the surrounding tissue start to soften before it is exposed to the MMP inhibitor.

Preferably, the present invention should be applied primarily on tendon and during intestine surgery. Other applications are also possible. For example, the present invention may be used for simple skin suturing. Additionally, the suture threads coated may be of a resorbable material.

The invention will now be illustrated by description of the drawings and non-limiting specific embodiments.

### DESCRIPTION OF EMBODIMENTS

First, the invention is illustrated with reference to the FIGS. 1 and 2.

FIG. 1 is a schematic cross-sectional view of the coated suture. Preferably, the suture thread 10 has a linker molecule layer 11 bound to the surface of the suture. A plurality of protein layers 12 such as fibrinogen layers are then applied on top thereof. A free carboxyl terminal of the first protein layer 12a may be activated by for example a carbodiimide, such as ethyl-dimethyl-aminopropylcarbodiimide (EDC), and hydroxy-succinimide (NHS) to attract and, by peptide bond formation, capture more protein so as to form a second protein layer 12b. The EDC activates the carboxyl groups, of the first protein layer, so that amino groups of the protein in solution may be chemically bound thereto. By repetition of the EDC/NHS activation procedure, a plurality of protein layers may be immobilized and cross-linked, so forming a matrix structure. The total thickness of the protein layers 12 may be increased by increasing the number of layers. The top layer 16 preferably include the enzyme inhibitor, such as an MMP inhibitor, so that a first amount of the inhibitor is covalently, or by other mechanisms firmly, bound to the protein and a second amount of the inhibitor is adhered to the protein by for example, absorption or any other mechanism which makes the release relatively easy.

FIG. 2 illustrates some of the steps used to coat the suture. In a first cleaning step 20, the suture is cleaned in for example a suitable alcohol. The suture surface is then hydrolyzed in a hydrolyze step 22. Charged and chemically reactive groups are created on the suture surface in a treatment step 24 and surface roughening is achieved at the same time in the nm scale if radio frequency plasma treatment is used for the generation of the reactive groups. The treatment step 24 may also include other physical as well as chemical and mechanical treatments to roughen the suture surface, if desired. In a binding step 26, a suitable linker molecule is then chemically bound to the suture surface, if desired. In an attachment step 28, the first protein layer is chemically attached to the suture surface or the linker molecule. In an adding step 30, a plurality of protein layers are added according to the principles described above. In an association step 32, a first amount of a suitable enzyme inhibitor is chemically bound to the protein layers by e.g. the EDC/NHS coupling chemistry described, or any other suitable method. A second amount of the enzyme inhibitor is absorbed in the matrix structure formed by the protein layers. Examples of the details of each step are illustrated in the examples below.

### SORT DESCRIPTION OF THE DRAWINGS

FIG. 1. is a schematic cross-sectional view of a coated suture of the invention

FIG. 2 is a block diagram showing some of the steps used to coat a suture of the invention.

FIG. 3 shows two diagrams of anastomotic strength of the rat colon on the third postoperative day. Doxycycline-coated sutures increased the breaking strength (a) by 17% (p=0.026) and the energy uptake at failure (b) by 20% (p=0.047). Data shown as mean (thick horizontal line) and s.d. interval. Filled circles, doxycycline-coated sutures; open circles, carrier-coated sutures. *p<0.05.

FIG. 4 shows a diagram regarding peak force at suture pull-out after tendon suture fixation with control and doxycycline-coated suture on days 0 or 3 after operation. Median and interquartile range. * one-tailed Mann-Whitney U test, p=0.028.

### Example 1

### Coating of Suture Material with EDC/NHS Crosslinked Fibrinogen and MMP-Inhibitor

Suture materials made of e.g. polyamides such as nylon-6,6 and nylon-6, or poly(p-dioxanone) or polylactide/-glycolide, are cleaned according to standard laboratory practice for 10 minutes by incubation in 70% ethanol followed by copious rinsing in distilled water and dried in nitrogen gas followed by 30 seconds exposure to UV. The structure surfaces become hydrolyzed during typically 3 hours in distilled water and treated one minute in a Radio Frequency Plasma chamber. Radio frequency plasma treatment roughens the surface of the suture material and generates charged and chemically reactive surface groups onto which for example spacers or proteins can be covalently attached. For example, surface carboxyl or amine groups may be formed on the suture via the surface activation procedures.

Thereafter, a linker molecule such as glutaraldehyde or ethyl-dimethyl-aminopropylcarbodiimide (EDC) is bound to the surface. One layer of fibrinogen from 1 mg/ml solution becomes covalently attached by the assistance of the linker molecule. More fibrinogen may subsequently be bound to this first layer in order to create a controllable but thin (thickness less than one micrometer) matrix into which the drug can be attached and/or associated.

Sutures with ten layers of fibrinogen may be prepared in the following way. Sutures prepared as above are then incubated for thirty minutes in 1 mg/ml protein dissolved in phosphate buffered saline (PBS) at pH 7.4. The specimen surfaces are thereafter extensively rinsed in PBS and incubated for thirty minutes in PBS at pH 5.5, containing 0.2M ethyl-dimethyl-aminopropylcarbodiimide (EDC). The specimen surfaces are again incubated for thirty minutes in a newly made 1-mg/ml protein solution in PBS, pH 5.5, thereafter rinsed in the PBS buffer and again incubated in the EDC/NHS solution. This procedure is repeated ten times to produce the ten-layer fibrinogen coating but is not limited to this number of protein incubations. Since the EDC/NHS solution is unstable at room conditions, new solutions are prepared every second hour.

The MMP-inhibitor, e.g. a tetracycline, is immobilized to the fibrinogen multilayer using the above-described EDC/NHS coupling technique. The suture specimens are stored in a solution of the same or a different MMP-inhibitor for up to 24 hours to allow additional loading of the matrix with loosely bound substance. The specimens are removed from the solution, blown dry in nitrogen, and kept sealed at ambient until used.

The thickness of the cross-linked fibrinogen layer is approximately 280 Angstroms and the MMP-inhibitor layers between 5 and 100 Ångstroms. The MMP-inhibitor coated suture interferes with MMP at the surgical site, lowering the activity of the latter. The gradual release of the MMP-inhibitor provides a sustained effect resulting in maintained integrity of the otherwise degenerated tissue, for example collagen and tendon that surrounds the suture threads.

### Example 2

### Alleviation of Postoperative Weakening of Sutured Intestinal Tissue by Use of a Matrix Metalloproteinase Inhibitor Coating on Sutures

Doxycycline was used in the experiment as MMP inhibitor for local delivery at the suture site since several experimental studies have shown beneficial effects of treatment with systemic MMP-inhibitors, e.g. doxycycline, most notably on the critical third postoperative day^{7,8}.

### Materials and Methods

### Suture Coating

Sterile 6-0 polybutester monofilament sutures (Novafil, Tyco Healthcare, Schaffhausen, Switzerland) were activated during 10 seconds on each side in a radio frequency plasma chamber (Plasmaprep 100; Nanotech, Sweden). The activated sutures were incubated for 30 minutes in 6% glutaraldehyde in phosphate buffered saline (PBS), pH 9. The surfaces were extensively rinsed in PBS, pH 9. Ten layers of fibrinogen (HYPHEN BioMed, Neuville-sur-Oise, France; molecular weight: 340 kDa, clotability 98%) were prepared as follows⁹: the glutaraldehyde coated sutures were incubated for 30 minutes in 1 mg/ml fibrinogen dissolved in PBS at pH 7.4. The sutures were extensively rinsed in PBS followed by incubation during 30 minutes in PBS, pH 5.5, containing 0.2 M ethyl-dimethyl-aminopropylcarbodiimide (EDC; Sigma-Aldrich, St. Louis, Mo., USA) and 0.05 M N-hydroxy-succinimide (NHS; Sigma-Aldrich). Then a new 1 mg/ml fibrinogen solution was prepared in PBS buffer, pH 5.5, and the sutures incubated for 30 minutes in this, rinsed in PBS buffer, and again incubated in the EDC/NHS solution. As the EDC solution is unstable at room conditions, new solutions were prepared every second hour. This procedure was repeated until ten fibrinogen layers were immobilised. The crosslinked fibrinogen surface was subsequently incubated in EDC/NHS as above, and for 3 hours in a 1 mg/ml solution doxycycline (Sigma-Aldrich) or for 3 hours in PBS (control sutures), and finally rinsed in distilled water.

Thicknesses of the fibrinogen and doxycycline layers on the sutures were measured by null ellipsometry (Auto-Ell III; Rudolph Research, Flanders, N.J., USA) in air, calculated according to the McCrackin evaluation algorithm¹⁰ and converted into an approximate adsorbed amount per unit area by de Feijter's formula¹¹. The assumed refractive index of the protein and immobilised doxycycline film was n_{f}=1.465¹². During the measurements, 1 nm of adsorbed proteins equalled approximately 120 ng/cm^{13,14}. By this method it was estimated that the total fibrinogen layer thickness was approximately 30 nm and the immobilised amount of doxycycline was approximately 240 ng/cm². The diameter of the uncoated suture threads was 0.095 mm. Therefore it could be calculated that 1 cm of the doxycycline-coated sutures carried about 7 ng of doxycycline.

The so aseptically prepared sutures were stored at room temperature in dark in a 0.5 mg/ml doxycycline PBS solution, pH 5.5, until use within 6 days. Fibrinogen-coated control sutures were stored in PBS, pH 5.5, under identical conditions without doxycycline. Doxycycline-coated and control sutures were indistinguishable by visual inspection and physical handling such as elasticity and pliability.

### Design and Surgical Procedure

Forty male Sprague Dawley rats (Taconic M&B, Ry, Denmark) weighing 330(27) g (mean (s.d.)) were randomised to three groups. Thirty rats received either doxycycline-coated (n=15) or carrier-coated sutures (n=15) and underwent biomechanical evaluation three days postoperatively. Additionally, the biomechanical properties were evaluated directly after the operation (immediate day 0 controls) of anastomoses constructed using carrier-coated sutures in 10 rats. One surgeon, blinded for treatment throughout the entire study period, performed all operations.

Anaesthesia was induced by a s.c. injection of a mixture of fentanyl citrate (0.16 mg/kg), droperidol (11.1 mg/kg) and midazolam (0.13 mg/kg). Rats were given carprofen (5 mg/kg s.c.) for analgesia. After laparotomy, a standardised 10 mm segment of the colon was resected 6 cm proximally to the anal orifice. An end-to-end anastomosis was constructed using 8 interrupted coated sutures placed approximately 2 mm from the resection margin. The abdomen was closed with continuous polyglactin suture in the musculofascial layer and metal clips in the skin. The animals were allowed immediate mobilisation and free access to water and pellets.

This study was approved by the Danish National Experimental Animal Inspectorate and followed the established guidelines.

### Mechanical Testing

After sacrifice, the abdomen was opened and the colon carefully freed from adhesions. The anastomosis segment was removed and gently cleaned of faecal contents. The segment, with the anastomosis in the middle, was mounted in a materials testing machine (LF Plus; Lloyds Instruments, Southampton, UK) with 10 mm between the clamps and stretched at a constant deformation rate of 10 mm/min until breaking. The maximal load (breaking strength) and the area under the curve to the breaking point (energy uptake) were derived from the load-strain curve calculated by the software (Nexygen; Lloyds Instruments). The breaking point was defined by the maximum force value. These measurements were carried out by an individual unaware of treatment.

### Statistics

The main hypothesis was that there would be a difference at 3 days postoperatively. Therefore, a sequential analysis of data was employed, as predetermined, to first evaluate the effect of doxycycline treatment at three days. Subsequently, the early decrease of anastomosis breaking strength was analysed, comparing immediate and day 3 controls. Student's t-test (two-tailed) was used. p<0.05 was considered statistically significant. Results are presented as mean (s.d).

### Results

Three rats in the control group and one rat in the doxycycline group died immediately after the operation, probably due to the anaesthesia. No difference in postoperative body weight gain was found between rats treated with doxycycline-coated sutures and rats treated with carrier-coated sutures.

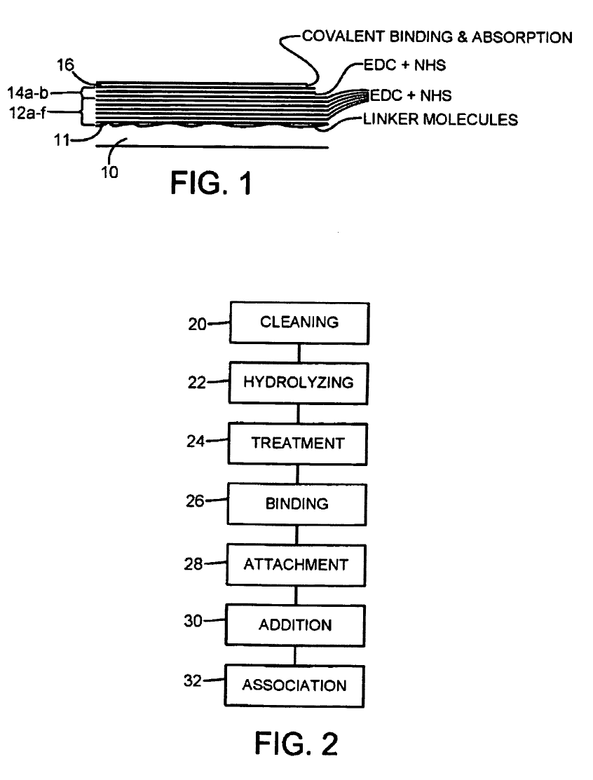

Breaking strength on the third day was higher in the doxycycline-coated sutures group (1.47(0.2) N) compared with the carrier-coated control group (1.25(0.3) N; p=0.026; FIG. 1a). The difference was 17% (95% Cl: 2 to 31%). Energy uptake was also higher in the doxycycline-coated sutures group (12.1(2.2) Nmm) compared with the carrier-coated control group (10.1(2.7) Nmm; p=0.047; FIG. 1b). The difference was 20% (95% Cl: 0 to 39%).

The breaking strength in the immediate day 0 control group was 1.53(4.4) N. Thus, the carrier-coated control group decreased 18% as compared with immediate day 0 control group (95% Cl: −2 to 39%; p=0.08). Local doxycycline treatment aborted roughly three quarters of this decrease at 3 days. The energy uptake in the immediate day 0 control group was 11.4(3.1) Nmm. Thus, the carrier-coated control group decreased by 11% as compared with the immediate controls (95% Cl: −11 to 34%; p=0.30). This decrease appeared totally aborted by local doxycycline treatment.

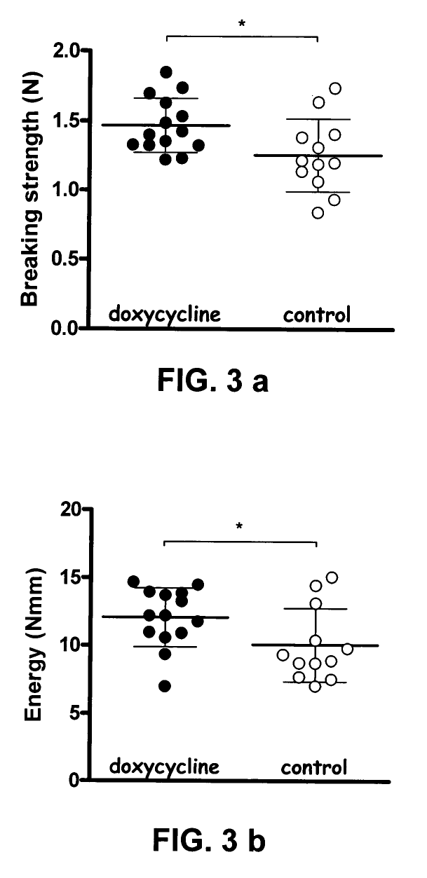

The results are illustrated in FIG. 3

This Example shows that local drug delivery via the sutures is as efficient as systemic MMP-inhibitor administration in this experimental model, but with the major advantage that drug doses are minimal.

### Example 3

### Improved Tendon Suture Fixation by Use of a Matrix Metalloproteinase Inhibitor Coating on Sutures

The effect of the MMP-inhibitor doxycycline on tendon suture fixation is evaluated in this experiment. First the effect of systemic doxycycline was examined. Then, doxycycline was applied locally, by using a coating comprising doxycycline on the suture material. Systemic and local doxycycline treatment were evaluated regarding suture pull-out strength in the rat Achilles tendon.

### Materials and Methods

### Systemic Treatment

40 male Sprague Dawley rats weighing 405 (SD 24) g were randomised to receive deionised drinking water with or without doxycycline 80 mg/kg/day, which gives a clinically relevant serum concentration. Suture fixation was evaluated 3 days after operation.

### Local Treatment

A nylon suture was coated with doxycycline hyclate (4 nm, Sigma) on top of EDC/NHS crosslinked fibrinogen (30 nm, Haemochrom Diagnostica). The total amount of immobilized drug was approximately 480 ng/cm². Regular nylon sutures served as controls.

43 male Sprague Dawley rats weighing 375 (SD 23) g were randomised to 3 groups: 16 controls and 17 rats receiving a doxycycline-coated suture were evaluated 3 days after operation. 10 more rats were evaluated immediately.

### Surgical Procedure and Mechanical Evaluation

The study was approved by the regional animal ethics committee. Animals were anaesthetised with isoflourane gas. An oblique 1 cm skin incision was made over the left Achilles tendon. A 3-0 monofilament nylon suture with a tapered needle (Dermalon, Tyco Healthcare) was inserted into the intact tendon to make a modified Kessler stitch spanning 1 cm longitudinally, starting at the tendon's superior end 2 mm from the musculotendinous junction. Thereafter the free ends were approximated with a double knot, leaving a loop 1 cm in diameter. The Achilles tendon was then cut transversely just proximally to the suture to unload the tendon. Thus, the Kessler stitch was only inserted into the distal part of the tendon. The suture was inserted into the tendon before transection to avoid unnecessary damage during handling. The plantaris tendon was cut and the skin was sutured.

On the evaluation day, the tendon with the attaching calcaneus was dissected clean from surrounding tissue. The calcaneus was fixed in a clamp while the suture loop was attached to a hook via a freely movable metal device to allow a straight pull. The complex was mounted in a materials testing machine and pulled at a constant speed of 0.1 mm/s until pull-out. Peak force was recorded. The two separate studies were analysed by way of Mann-Whitney U tests. Because systemic treatment indicated a positive effect, a one-tailed test was used for local treatment.

### Results

### Systemic Treatment

Two rats from the control group and six rats from the doxycycline group were excluded from analysis either due to technical issues or weight loss >2SD below mean weight loss.

The suture pull-out strength was higher in the doxycycline treated tendons (median 16 N, interquartile range 14-20) as compared to controls (median 14 N, interquartile range 12-16) on the third postoperative day (p=0.025).

### Local Treatment

One rat died postoperatively (control 3 d group) and three rats were excluded from analysis due to technical problems (one rat from the 0 d control group and two rats from the doxycycline group).

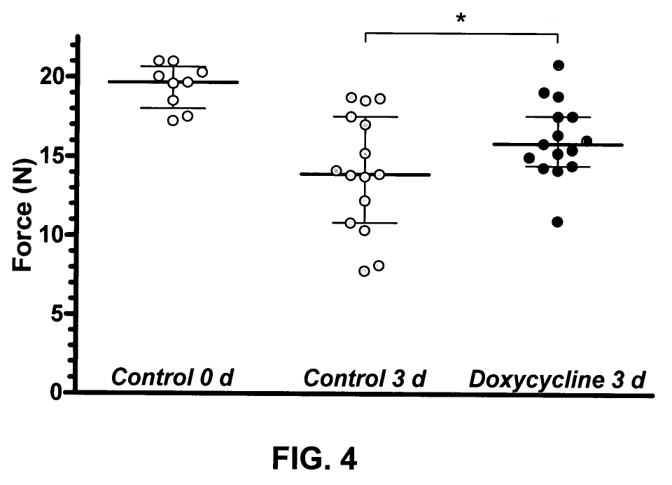

The suture pull-out strength was higher in doxycycline treated tendons as compared to controls on the third postoperative day (p=0.028) (FIG. 4). The median suture pull-out strength in the controls at three days was decreased by 5.8 N. Doxycycline treatment aborted 34% of this decrease.

This example indicates that tendon suture fixation can be improved by treatment with an MMP-inhibitor, and the size of the effect was similar with both systemic and local administration. Further, local doxycycline aborted a third of the unwanted decrease in suture fixation after three days.

While the present invention has been described in accordance with preferred compositions and embodiments, it is to be understood that certain substitutions and alterations may be made thereto without departing from the spirit and scope of the following claims. The teachings of the cited articles are included herein by reference.

### REFERENCES

1. McDowell C L et al. J Hand Surg [Am] 2002; 27:605.
2. Peterson J T. Matrix metalloproteinase inhibitor development and the remodeling of drug discovery. Heart Fail Rev 2004; 9: 63-79.
3. Mirastschijski U, Haaksma C J, Tomasek J J, Agren M S. Matrix metalloproteinase inhibitor GM 6001 attenuates keratinocyte migration, contraction and myofibroblast formation in skin wounds. Exp Cell Res 2004; 299: 465-475.
4. Chambers W M, Mortensen N J. Postoperative leakage and abscess formation after colorectal surgery. Best Pract Res Clin Gastroenterol 2004; 18: 865-880.
5. Stumpf M, Klinge U, Wilms A, Zabrocki R, Rosch R, Junge K et al. Changes of the extracellular matrix as a risk factor for anastomotic leakage after large bowel surgery. Surgery 2005; 137: 229-234.
6. Shaper K R, Savage F J, Hembry R M, Boulos P B. Regulation of matrix metalloproteinases in a model of colonic wound healing in a rabbit. Dis Colon Rectum 2001; 44: 1857-1866.
7. Ågren MS, Jorgensen L N, Delaissé J M. Matrix metalloproteinases and colon anastomosis repair: a new indication for pharmacological inhibition? Mini Rev Med Chem 2004; 4: 769-778.
8. Siemonsma M A, de Hingh I H, de Man B M, Lomme R M, Verhofstad A A, Hendriks T. Doxycycline improves wound strength after intestinal anastomosis in the rat. Surgery 2003; 133: 268-276.
9. Tengvall P, Jansson E, Askendal A, Thomsen P, Gretzer C. Preparation of multilayer plasma protein films by EDC/NHS coupling chemistry on silicon. Coll Surf B: Biointerfaces 2003; 28: 261-272.
10. McCrackin F L. A FORTAN Program for the Analysis of Ellipsometer Measurements. In: NBS Technical Note: Washington D.C., 1969; 479.
11. de Feijter J A, Benjamins J, Veer F A. Ellipsometry as a tool to study the adsorption of synthetic and biopolymers at the air-water interface. Biopolymers 1978; 17: 1759-1773.
12. Benesch J, Askendal A, Tengvall P. Quantification of adsorbed human serum albumin at solid interfaces: a comparison between radioimmunoassay (RIA) and simple null ellipsometry. Coll Surf B: Biointerfaces 2000; 18: 71-81.
13. de Hingh, de Man B M, Lomme R M, van Goor H, Hendriks T. Colonic anastomotic strength and matrix metalloproteinase activity in an experimental model of bacterial peritonitis. Br J Surg 2003; 90: 981-988.
14. Stenberg M, Nygren H. The Use of the Isoscope Ellipsometer in the Study of Adsorbed Proteins and Biospecific Binding Reactions. J dé Physique 1983; C10 (S12); 83-86.

1. A coated suture thread comprising an immobilized and crosslinked fibrinogen matrix into and/or onto which one or several pharmacological substance(s) that inhibit tissue break-down is (are) attached and/or associated.

2. The coated suture thread according to claim 1, wherein the matrix is formed on a rough surface of the thread.

3. The coated suture thread according to claim 1, wherein the matrix is covalently coupled to the suture thread surface.

4. The coated suture thread according to claim 3, wherein the covalent coupling of the matrix to the suture thread surface is via a linker molecule.

5. The coated suture thread according to claim 4, wherein the linker molecule is glutaraldehyde.

6. The coated suture thread according to claim 1, wherein the fibrinogen matrix is built up of several fibrinogen layers selected from 2 to 20 layers.

7. The coated suture thread according to claim 1, wherein the one or several pharmacological substance(s) is (are) selected from the group consisting of tetracyclines, chemically modified tetracyclines, synthetic matrix metalloproteinase inhibitors, including those of the hydroxamate subgroup; cyclooxygenase inhibitors, including cyclooxygenase 2 specific inhibitors; nuclear factor kappa B inhibitors; lipooxygenase inhibitors; corticosteroids including glucocorticoids; macrolide antibiotics; hydroxymethylglutaryl coenzyme A reductase inhibitors (statins); angiotensin converting enzyme (ACE) inhibitors; angiotensin II receptor blockers (ARBs); bisphosphonates; aprotinin; gabexate mesilate; sulfasalazine; inhibitors of tumour necrosis factor alpha;
and transforming growth factor beta inhibitors.

8. The coated suture thread according to claim 1, wherein one pharmacological substance is a matrix metallo-proteinase inhibitor (MMP-inhibitor).

9. The coated suture thread according to claim 1, wherein one pharmacological substance is a corticosteroid.

10. The coated suture thread according to claim 1, wherein one pharmacological substance is a cyclooxygenase inhibitor (COX-inhibitor).

11. A method of producing a coated suture thread comprising the steps of immobilizing a first layer of fibrinogen onto a suture thread surface to be coated,
crosslinking a second layer of fibrinogen to the first layer of fibrinogen to form a fibrinogen matrix,
optionally increasing the number of layers of the fibrinogen matrix by crosslinking one or several layers of fibrinogen on top of the second layer of fibrinogen, and attaching and/or associating one or several pharmacological substance(s) that inhibit tissue break-down into and/or onto the matrix of immobilized and crosslinked plurality of fibrinogen layers.

12. The method of producing a coated suture thread according to claim 11, wherein the surface to be coated is roughened to increase the surface area thereof.

13. The method of producing a coated suture thread according to claim 12, wherein the surface to be coated is treated to generate chemically reactive groups thereon.

14. The method of producing a coated suture thread according to claim 1, wherein the first fibrinogen layer is covalently coupled to the suture thread surface.

15. The method of producing a coated suture thread according to claim 1, wherein the covalent coupling of the first fibrinogen layer to the suture thread surface is accomplished by covalently coupling one end of a linker molecule to the suture thread surface and the other to the first layer of fibrinogen.

16. The method of producing a coated suture thread according to claim 15, wherein the linker molecule is glutaraldehyde.

17. The method of producing a coated suture thread according to claim 1, wherein the total number of fibrinogen layers of the fibrinogen matrix produced is selected from 2 to 20 layers.

18. The method of producing a coated suture thread according to claim 1, wherein the one or several pharmacological substance(s)) that inhibit tissue break-down is (are) selected from the group consisting of tetracyclines, chemically modified tetracyclines, synthetic matrix metalloproteinase inhibitors, including those of the hydroxamate subgroup; cyclooxygenase inhibitors, including cyclooxygenase 2 specific inhibitors; nuclear factor kappa B inhibitors; lipooxygenase inhibitors; corticosteroids including glucocorticoids; macrolide antibiotics; hydroxymethylglutaryl coenzyme A reductase inhibitors (statins); angiotensin converting enzyme (ACE) inhibitors; angiotensin II receptor blockers (ARBs); bisphosphonates; aprotinin; gabexate mesilate; sulfasalazine; inhibitors of tumour necrosis factor alpha; and transforming growth factor beta inhibitors.

19. The method of producing a coated suture thread according to claim 18, wherein one selected pharmacological substance is a matrix metallo-proteinase inhibitor (MMP-inhibitor).

20. The method of producing a coated suture thread according to claim 18, wherein one selected pharmacological substance is a corticosteroid.

21. The method of producing a coated suture thread according to claim 18, wherein one selected pharmacological substance is a cyclooxygenase inhibitor (COX-inhibitor).

22. A method of treating a subject in need of suturing damaged tissue comprising suturing the damaged tissue with a suture thread according to claim 1.

23. The method according to claim 22, wherein the damaged tissue is selected form tendon, ligament, intestine and skin.
